(19) United States
(12) Patent Application Publication
(10) Pub. No.: US 2007/0135923 A1
(43) Pub. Date: Jun. 14, 2007

(54) Ceramic and polymer prosthetic device

(75) Inventors: Marc M. Peterman, Austin, TX (US); Shannon M. Vittur, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/303,085

(22) Filed: Dec. 14, 2005

(51) Int. Cl. A61F 2/44

(52) U.S. Cl. 623/17.14; 623/17.15

(57) ABSTRACT

A prosthetic device for insertion into an intervertebral space may include a first articulating element formed of a ceramic material and a second articulating element configured to cooperate with the first articulating element to permit articulating motion. The second articulating element also may be formed of a ceramic material. A first polymer component may be joined to the first articulating element at a first ceramic-polymer interface and a second polymer component may be joined to the second articulating element at a second ceramic-polymer interface. A method of manufacturing the disc is also disclosed.

### BACKGROUND

Implantation of an articulating disc is one way of treating injured, degraded or diseased spinal joints. Some articulating discs incorporate low-friction ceramic surfaces. Because ceramics tend to be brittle, and a single crack could cause a catastrophic failure, a typical conventional disc includes a metal backing for the ceramic that imparts sturdiness and supports the ceramic surfaces. The metal backing, although it may be treated to promote bone growth, typically interfaces with bone structure, such as vertebral endplates. However, over time, as the hard metals interface with the bone structure, resorption response or other bone degradation may occur. In addition, the metal backing can be overly stiff, subjecting the ceramic components to high stress. This stress can initiate brittle and catastrophic failure of the ceramic components.

What is needed is prosthetic device that prolongs the life of ceramic articulating members. The intervertebral prosthetic disc disclosed herein overcomes at least one of the disadvantages of the prior art.

### SUMMARY

In one exemplary aspect, this disclosure is directed to a prosthetic device for insertion into an intervertebral space. The prosthetic device may include a first articulating element formed of a ceramic material and a second articulating element configured to cooperate with the first articulating element to permit articulating motion. The second articulating element also may be formed of a ceramic material. A first polymer component may be joined to the first articulating element at a first ceramic-polymer interface and a second polymer component may be joined to the second articulating element at a second ceramic-polymer interface.

In another exemplary aspect, this disclosure is directed to another prosthetic device for insertion into an intervertebral space formed between upper and lower vertebral bodies. The prosthetic device may include a first articulating element formed of a ceramic material and a second articulating element configured to cooperate with the first articulating element to permit articulating motion. The second articulating element also may be formed of a ceramic material. A first polymer endplate may be molded to the first articulating element at a first ceramic-polymer interface. The first polymer endplate may have an upper surface configured to contact the upper vertebral body at a first vertebra-polymer interface. A second polymer endplate may be molded to the second articulating element at a second ceramic-polymer interface. The second polymer endplate may have a lower surface configured to contact the lower vertebral body at a second vertebra-polymer interface.

In yet another exemplary aspect, this disclosure is directed to a method of forming a prosthetic device for insertion into an intervertebral space formed between upper and lower vertebral bodies. The method may include manufacturing a first articular component by placing a first articulating element formed of a ceramic material into a mold. A polymer material may be introduced into the mold. The first articulating element and the polymer material may be compressed to mold the polymer to the first articulating element to create a first ceramic-polymer interface and to form the first articular component. The method also may include manufacturing a second articular component by placing a second articulating element formed of a ceramic material into a mold. Again, a polymer material may be introduced into the mold. The second articulating element and the polymer material may be compressed to mold the polymer to the second articulating element to create a second ceramic-polymer interface and form the second articular component. The first and second articulating elements may be configured to cooperate to provide articulation to the prosthetic device.

### BRIEF DESCRIPTION OF THE DRAWINGS

FIG. 1 is a pictorial representation of a lateral view of a portion of a vertebral column.

FIG. 2 is a pictorial representation of a lateral view of a pair of adjacent vertebral bodies defining an intervertebral space.

FIG. 3 is a pictorial representation of a perspective view of one exemplary intervertebral prosthetic disc.

FIG. 4 is a pictorial representation of a cross-sectional view of the exemplary intervertebral prosthetic disc of FIG. 3 between vertebral bodies.

FIG. 5 is a pictorial representation of a cross-sectional view of the exemplary intervertebral prosthetic disc of FIG. 3 in an articulating state.

FIG. 6 is a pictorial representation of a cross-sectional view of an alternate exemplary intervertebral prosthetic disc.

FIGS. 7-9 are pictorial representations of cross-sectional views of additional exemplary embodiments of intervertebral prosthetic discs.

FIGS. 10 and 11 are pictorial representations of exemplary endplates of an intervertebral prosthetic disc.

FIGS. 12-14 are pictorial representations of cross-sectional views of exemplary endplates of an intervertebral prosthetic disc

### DETAILED DESCRIPTION

The present invention relates generally to vertebral reconstructive devices, and more particularly, to an articular intervertebral prosthetic disc for implantation. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

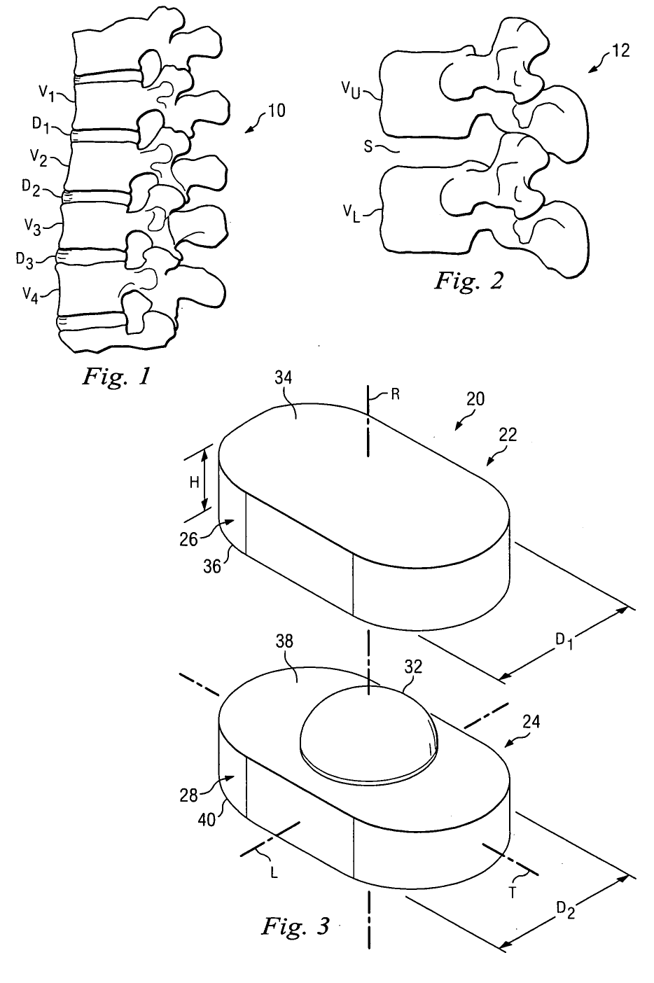

FIG. 1 shows a lateral view of a portion of a spinal column 10, illustrating a group of adjacent upper and lower vertebrae V1, V2, V3, V4 separated by natural intervertebral discs D1, D2, D3. The illustration of four vertebrae is only intended as an example. Another example would be a sacrum and one vertebrae.

For the sake of further example, two of the vertebrae will be discussed with reference to FIG. 2. The two vertebrae form a spinal segment 12 including a lower vertebrae V_{L} and an upper vertebrae V_{U}. During a disc arthroplasty procedure, some or all of the natural disc positioned between the two vertebrae V_{L}, V_{U} may be removed via a discectomy or a similar surgical procedure. Removal of the diseased or degenerated disc results in the formation of an intervertebral space S between the upper and lower vertebrae V_{U}, V_{L}, as shown in FIG. 2

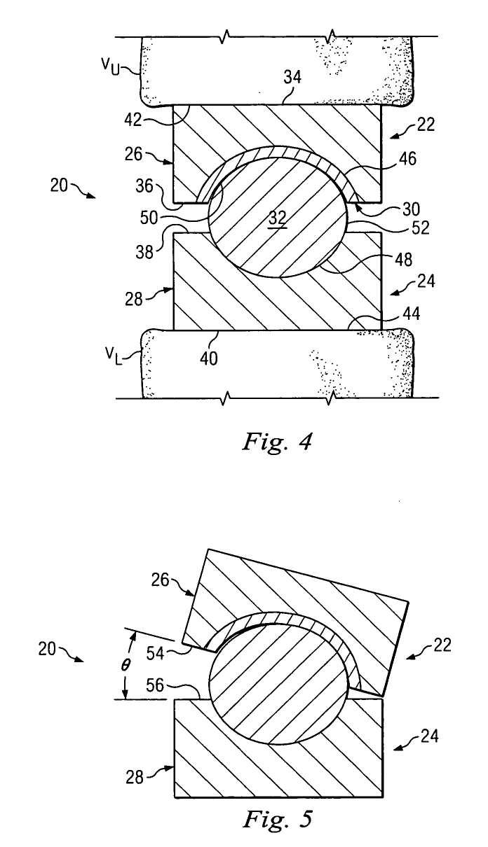

FIGS. 3 and 4 show one exemplary embodiment of an intervertebral prosthetic disc 20 for insertion into the intervertebral space S of FIG. 2. The prosthetic disc 20 includes an upper articular component 22 and a lower articular component 24. The designation of “upper” and “lower” is used for descriptive purposes only, as the prosthetic disc 20 may be flipped so that that articular component 22 is the lower component and the articular component 24 is the upper component.

The upper articular component 22 and the lower articular component 24 of the prosthetic disc 20 may provide relative pivotal and rotational movement between the adjacent vertebral bodies to maintain or restore motion substantially similar to the normal bio-mechanical motion provided by a natural intervertebral disc. More specifically, the articular components 22, 24 may be configured to pivot relative to one another about a number of axes, including lateral or side-to-side pivotal movement about longitudinal axis L and anterior-posterior pivotal movement about transverse axis T. In some embodiments, the articular components 22, 24 are permitted to pivot relative to one another about any axis that lies in a plane that intersects longitudinal axis L and transverse axis T. Furthermore, the articular components 22, 24 may be configured to rotate relative to one another about a rotational axis R. It should be understood that other combinations of articulating movement are also possible, such as, for example, relative translational or linear motion, and such movement, among other movement directions, is contemplated as falling within the scope of the present disclosure.

The upper and lower articular components 22, 24 each respectively include upper and lower endplates 26, 28 and upper and lower articulating elements 30, 32, best seen in FIG. 4. Conventional prosthetic discs typically include endplates formed of a metal material to provide support and backing to the articulating elements. The metal endplates, although perhaps treated with ingrowth material or other substances, are in contact with and support the upper and lower vertebrae. Although shaped and prepared before implantation of the disc, the bony plate surfaces of the upper and lower vertebrae seldom exactly conform in profile to the endplates of the articular components. Because of this, high spots on the bony surface typically carry spinal loads, rather than the loads being evenly distributed over the hard metal endplates. Because the metal endplates have little flexion or ability to conform, the bony high spots receive all the stress applied during normal daily routines. In response to the high spots contacting the metal endplates, the bony vertebral plates often begin their own degradation and resorption.

In order to be more compatible with the bony vertebrae, the upper endplate 26 and the lower endplate 28 of the prosthetic disc 20 disclosed herein may be formed of a polymer material, rather than a metal material. In some exemplary embodiments, the upper and lower endplates 26, 28 are formed of polymers selected from the polyaryletherketone (PAEK) family. For example, the upper and lower endplates 26, 28 may be formed of, for example, polyetheretherketone (PEEK), carbon-reinforced PEEK, or polyetherketoneketone (PEKK). In other embodiments, the upper and lower endplates 26, 28 may be formed of polysulfone, polyetherimide, polyimide, ultra-high molecular weight polyethylene (UHMWPE), or cross-linked UHMWPE, among other polymers. In some embodiments, the polymer material forming the upper and lower endplates is reinforced, while in other embodiments, the polymer material is unreinforced, or consists substantially of the polymer material.

The upper endplate 26 may include a top surface 34 and a bottom surface 36. The top surface 34 may be configured to interface with a lower surface of the upper vertebrae shown in FIG. 4. Accordingly, the top surface 34 and the upper vertebra may form an upper polymer-bone interface 42 (FIG. 4). Similarly, the lower endplate 28 may include a top surface 38 and a bottom surface 40. The lower endplate 28 may contact the lower vertebrae of FIG. 4 with the bottom surface 40. Accordingly, the bottom surface 40 and the lower vertebrae form a lower polymer-bone interface 44 (FIG. 4). Some exemplary polymer-bone interfaces will be described in detail further below.

The upper articulating element 30 and the lower articulating element 32 may be formed of ceramic materials that engage each other to allow articulation. In some exemplary embodiments, alumina, zirconia, or a stabilized ceramic may be incorporated in the upper and lower articulating elements 30, 32. In the exemplary embodiment shown, the upper articulating element 30 includes a recessed-bearing surface 50, while the lower articulating element 32 includes a protruding-bearing surface 52. These recessed and protruding bearings surfaces define a ball-and-socket joint that provides articulation in any direction. The articulating elements could be shaped to provide articulation through joints other than a ball-and-socket style joint. For example, the articulating elements could form a trough and recess joint, a pea and saucer joint, or other joint imparting articulation to the prosthetic disc 20.

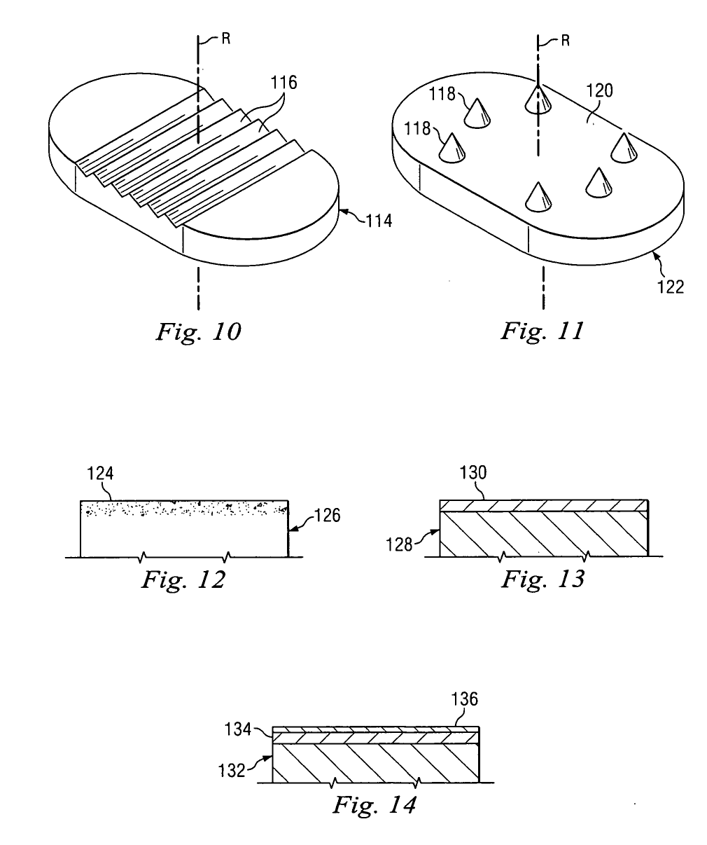

At least a part of the upper articulating element 30 may be embedded within the upper endplate 26, forming an upper ceramic-polymer interface 46, as shown in FIG. 4. In a similar manner, the lower articulating element 32 may be embedded within the lower endplate 28, forming a lower ceramic-polymer interface 48. In some examples, the articulating elements may be embedded in the endplates during a molding process. In one example, the ceramic articulating elements may be preformed using methods known in the art. Then, the articulating element may be roughened by grit blasting, sand blasting, or other roughening method. In some examples, only the side configured to bond with the polymer is roughened. The formed articulating elements may be placed within a polymer mold and polymer powder may be added to the mold. Under high temperature and pressure, the endplates may be formed while initiating a bond with the ceramic articulating elements. After removal of the molded polymer ceramic, the exposed ceramic surfaces may be polished to reduce friction when articulating against another ceramic surface. The polymer also may be treated with ingrowth coatings or material as discussed below with reference to FIGS. 12-14. In yet another exemplary embodiment, the polymer is preformed and adhered to the ceramic using an adhesive cement or other bonding element. Some additional specific embodiments of discs having ceramic-polymer interfaces are described in further detail below.

As shown in FIG. 5, the upper and lower endplates 26, 28 may each include a respective upper and lower shoulder 54, 56 extending outwardly from the respective upper and lower articulating elements 30, 32. Here, the shoulders 54, 56 are formed by the bottom surface 36 of the upper endplate 26 and the by the top surface 38 of the lower endplate 28. As best seen in FIG. 5, the upper shoulder 54 interacts with lower shoulder 56 to limit a range of articulation of the prosthetic disc 20. The range of articulation may be dictated by the size, the slope, and/or the shape of the shoulders 54, 56 and by the relative heights of the upper and lower articulating elements 30, 32 relative to the shoulders. In one exemplary embodiment, the range of articulation in one direction, designated θ in FIG. 5, is between about 5° and 20°. In another exemplary embodiment, the range of articulation in one direction is between about 12° and 15°. The prosthetic disc 20 could be configured to have other articulating angles as would be apparent to one skilled in the art.

In addition to limiting the range of articulation, the shoulders 54, 56 may protect the upper and lower articulating elements 30, 32 from contacting or impacting and impinging upon any additional component, such as the upper or lower endplates 26, 28 or the shoulders 54, 56. Instead, when articulation is at its limit, the shoulders 54, 56 contact each other as shown in FIG. 5. This may be helpful due to a potentially brittle nature of some ceramic components. Accordingly, because the shoulders 54, 56 contact each other first, the opportunity for the edge of the upper articulating element 30 to impinge upon the lower endplate 28 or the lower articulating element 32, even during maximum articulation, is reduced. This protects the upper and lower articulating elements 30, 32 from impact stresses that may otherwise arise.

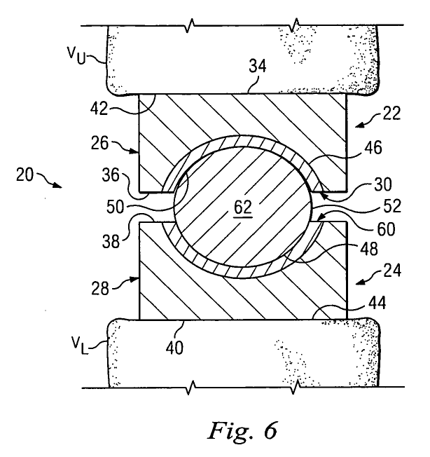

FIG. 6 shows an alternate embodiment of a prosthetic disc in accordance with the principles of the present invention. FIG. 6 varies from FIG. 4 in that the lower articulating component 24 includes a lower articulating element 60 that is formed of a ceramic material, as described above. Between the upper and lower articulating elements 30, 60, a nucleus 62 provides articulation to the upper and lower articulating components 22, 24. The nucleus 62 may be free-floating between the upper and lower articulating elements 30, 60 or may be attached in a known manner to one or both of the articulating elements 30, 60. The nucleus 60 may be formed of a ceramic or other material to provide low friction articulation.

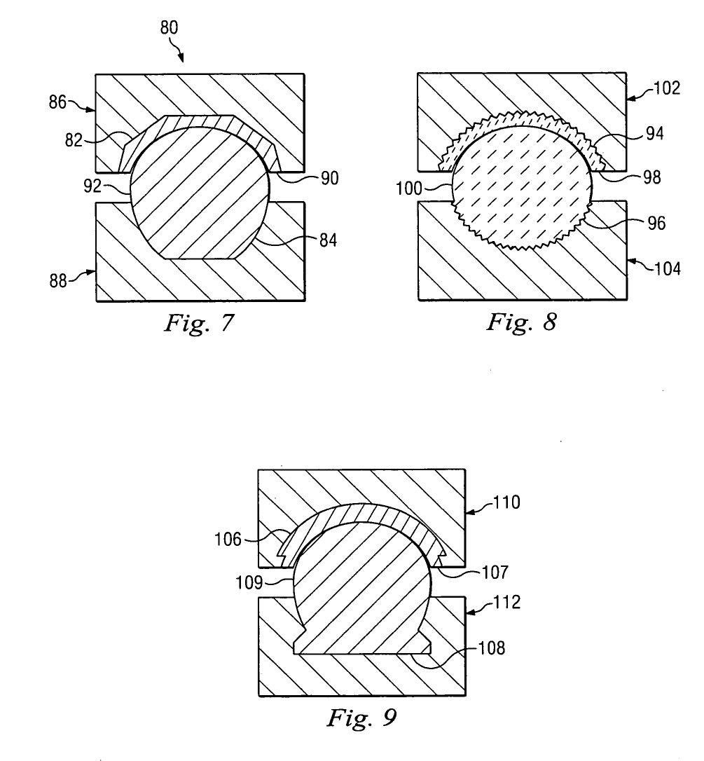

FIGS. 7-9 show alternative prosthetic discs having various ceramic-polymer interfaces. For example, with reference to FIG. 7, a prosthetic disc 80 includes an upper ceramic-polymer interface 82 and a lower ceramic-polymer interface 84. The ceramic-polymer interfaces 82, 84 are formed by upper and lower endplates 86, 88 in contact with upper and lower articulating elements 90, 92. In this example, the profiles of the articulating elements 90, 92 include a series of relatively straight lines connected at corners. Accordingly, the ceramic-polymer interfaces 82, 84 include the same series of relatively straight lines connected at comers. This increases the surface area of the interfaces 82, 84, may improve bonding, and may reduce the chance of the articulating elements 90, 92 separating from the endplates 86, 88. Other profiles also may be used to affect the interface surface area.

FIG. 8 shows upper and lower ceramic-polymer interfaces 94, 96 having ridges that increase the surface area of the interface and mechanically lock upper and lower articulating elements 98, 100 to respective upper and lower polymer endplates 102, 104. In some embodiments, the ridges are variations in height formed by surface roughening. Accordingly, the polymer material may flow into the ridges or roughened surfaces, again increasing the surface area of the interfaces 94, 69 and helping to affix together the ceramic and polymer materials.

In FIG. 9, upper and lower ceramic-polymer interfaces 106, 108 are formed by upper and lower ceramic articulating elements 107, 109 that each include ridges or indentations formed therein. The ridges or indentations receive a part of respective upper and lower polymer endplates 110, 112, thereby acting as a mechanical lock to secure the ceramic within the polymer endplate.

In addition to the exemplary embodiments shown, other exemplary embodiments are also contemplated. For example, the articulating elements may include threaded or waved surfaces that assist in securing the articulating elements into the endplates. Any increase in surface area may assist in securing the articulating element within the endplate and, therefore, may be desirable.

FIGS. 10 and 11 show exemplary mechanical features or means that may be included on or formed in the endplate of the prosthetic disc. These features may assist in securing the endplates to the vertebral bodies at the polymer-bone interfaces. While only the top surface of an upper endplate is shown and discussed, it is contemplated that the lower surface of a lower endplate may have the same or similar features. It should be noted, however, that the lower endplate also may have features varying from those of the upper endplate.

In FIG. 10, an upper endplate 114 includes a series of steps or ridges 116 that form high friction contact points when in contact with an upper vertebrae. The steps or ridges 116 may be tapered in one direction to facilitate insertion of the prosthetic disc into the intervertebral space between the upper and lower vertebrae while preventing removal of the prosthetic disc from the space.

FIG. 11 shows a number of protrusions 118 formed on a top surface 120 of an upper endplate 122. In the exemplary embodiment shown, the protrusions 118 are conical, pointed protrusions extending from the top surface 120. However, in other embodiments, the protrusions are shaped as spikes, screws, bumps, or as other protrusions that promote increased friction with a vertebra. Other suitable features may include ridges or keels, serrations, or diamond cut surfaces, fins, posts, and/or other surface features.

FIGS. 12-14 show exemplary treatments that may be included or formed in the endplate of the prosthetic disc. These treatments may assist in securing the endplates to the vertebral bodies at the polymer-bone interfaces. Again, while only the top surface of an upper endplate is shown and discussed, it is contemplated that the lower surface of a lower endplate may or may not have the same or similar features.

FIG. 12 shows an exemplary porous structure that may be formed at a top surface 124 of an upper endplate 126, the porous structure may enable bone growth and may increase interaction between the bone and the upper endplate 126. The porous structure may be formed in the upper endplate 126 using any known process. For example, a laser sintering process or a pore-forming gas process may be used. In some exemplary embodiments, the porous structure also includes a hydroxyapatite or tricalcium phosphate treatment that further aids in bone ingrowth.

FIG. 13 shows an exemplary embodiment of the upper endplate 128 having an applied ingrowth coating 130 that may enhance the fixation of the implanted prosthetic disc. For example, the surfaces may be roughened, and then the coating 130 may be applied by sintering by spraying, or other methods. In one example, the ingrowth coating 130 is a titanium plasma spray. However, other coatings could be used, including, for example, meshes, bead coatings, and beaded surfaces. Other types of coatings, including porous metal coatings, such as, for example, coatings of a trabecular metal, also may be used. In some examples the coating 130 may be a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. These coatings provide a substrate for ingrowth at the polymer-bone interface to attach the polymer to the bone. In some embodiments, HA, TCP, or other material, such as bone growth materials, may be applied to the polymer endplates as an integral part of the polymer. These may be applied using any known process, including a plasma spray or a vapor deposition process, and may have a structure similar to that used in bone phylic substrates. These materials may consist of highly crystalline or resorbable make-ups.

FIG. 14 shows an exemplary upper endplate 132 having a first coating 134 and a second coating 136 at the polymer-bone interface. The first coating may be a titanium plasma spray, while the second coating may be a treatment of hydroxyapatite (HA) or tricalcium phosphate (TCP). Other bone growth inducing substances could also be used, including those discussed above with reference to FIG. 13.

Using a polymer as upper and lower endplates and a ceramic as upper and lower articulating elements provides additional protection to the upper and lower vertebrae, i.e., the polymers may be strong enough to provide support to the brittle ceramics while still being soft enough to provide some cushioning and impact dampening to the vertebrae. Because the polymer is less hard than most metals, it can support the ceramic without introducing stress risers to the ceramic. This may increase the reliability of the disc and extend its total disc life. In addition, the polymer endplates are less stiff than some metals, and may prevent stress-induced resorption and degradation to the bone structure by providing a non-metal polymer-bone interface, while also providing some amount of cushioning and impact dampening. A reduction in resorption response may contribute to a stronger, less painful bone.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “rostral,” “caudal,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

We claim:

1. A prosthetic device for insertion into an intervertebral space, comprising:
a first articulating element formed of a first ceramic material;
a second articulating element configured to cooperate with the first articulating element to permit articulating motion, the second articulating element being formed of a second ceramic material;
a first polymer component joined to the first articulating element at a first ceramic-polymer interface; and
a second polymer component joined to the second articulating element at a second ceramic-polymer interface.

2. The prosthetic device of claim 1, wherein the first polymer component is molded onto the first articulating element during a compression molding process.

3. The prosthetic device of claim 1, wherein the first articulating element is joined to the first polymer component using an adhesive or cement.

4. The prosthetic device of claim 1, wherein the first articulating element includes a roughened surface extending along at least a portion of the first ceramic-polymer interface.

5. The prosthetic device of claim 1, wherein the first articulating element includes a profile having at least one of curved features and angled features that affect the surface area of the interface.

6. The prosthetic device of claim 1, wherein the first and second articulating elements form at least one of a ball-and-socket joint and a trough and recess joint.

7. The prosthetic device of claim 1, wherein the first and second polymer components each include a shoulder extending about the respective first and second articulating elements.

8. The prosthetic device of claim 7, wherein a degree of articulation is limited by the shoulders of the first and second polymer components.

9. The prosthetic device of claim 1, wherein the first and second polymer components are endplates, the first polymer component having an upper surface configured to engage an upper vertebral body at an upper vertebra-polymer interface, and the second polymer component having a lower surface configured to engage a lower vertebral body at a lower vertebra-polymer interface.

10. The prosthetic device of claim 9, wherein the upper surface includes at least one feature for mechanically engaging the upper vertebral body.

11. The prosthetic device of claim 10, wherein the at least one feature is one of teeth and spikes.

12. The prosthetic device of claim 9, wherein the upper surface is a porous structure that promotes bone ingrowth at the vertebra-polymer interface.

13. The prosthetic device of claim 9, wherein the upper surface includes a coating that promotes bone ingrowth at the vertebra-polymer interface.

14. The prosthetic device of claim 9, wherein the upper surface includes a bone ingrowth inducing material.

15. The prosthetic device of claim 9, wherein the first polymer component includes a porous metal that promotes bone ingrowth at the vertebra-polymer interface.

16. The prosthetic device of claim 1, wherein the first polymer component is formed of a polymer from the PAEK family of polymers.

17. The prosthetic device of claim 1, wherein the first and second ceramic materials are the same type of materials and include at least one of alumina, zirconia, and a stabilized ceramic.

18. The prosthetic device of claim 1, wherein the first articulating element includes a mechanical lock at the first ceramic-polymer interface to help secure the first articulating element to the first polymer component.

19. The prosthetic device of claim 1, wherein the first and second polymer components substantially consist of polymer material.

20. A prosthetic device for insertion into an intervertebral space formed between upper and lower vertebral bodies, comprising:
a first articulating element formed of a first ceramic material;
a second articulating element configured to cooperate with the first articulating element to permit articulating motion, the second articulating element being formed of a second ceramic material;
a first polymer endplate molded to the first articulating element at a first ceramic-polymer interface, the first polymer endplate having an upper surface configured to contact the upper vertebral body at a first vertebra-polymer interface; and
a second polymer endplate molded to the second articulating element at a second ceramic-polymer interface, the second polymer endplate having a lower surface configured to contact the lower vertebral body at a second vertebra-polymer interface.

21. The prosthetic device of claim 20, wherein the first polymer endplate is molded onto the first articulating element during a compression molding process.

22. The prosthetic device of claim 20, wherein the first and second articulating elements form at least one of a ball-and-socket joint and a trough and recess joint.

23. The prosthetic device of claim 20, wherein the first and second polymer endplates each include a shoulder extending about the respective first and second articulating elements, the shoulders being configured to limit a degree of articulation of the first and second polymer endplates.

24. The prosthetic device of claim 20, wherein the upper surface includes at least one feature for mechanically engaging the upper vertebral body.

25. The prosthetic device of claim 24, wherein the at least one feature is one of teeth and spikes.

26. The prosthetic device of claim 20, wherein the upper surface is a porous structure that promotes bone ingrowth at the vertebra-polymer interface.

27. The prosthetic device of claim 20, wherein the upper surface includes a coating that promotes bone ingrowth at the vertebra-polymer interface.

28. The prosthetic device of claim 20, wherein the upper surface includes a bone ingrowth inducing material.

29. The prosthetic device of claim 20, wherein the first polymer endplate includes a porous metal that promotes bone ingrowth at the vertebra-polymer interface.

30. The prosthetic device of claim 20, wherein the first polymer endplate is formed of a polymer from the PAEK family of polymers.

31. The prosthetic device of claim 20, wherein the first ceramic material includes at least one of alumina, zirconia, and a stabilized ceramic.

32. A method of forming a prosthetic device for insertion into an intervertebral space formed between upper and lower vertebral bodies, comprising:
manufacturing a first articular component by
placing a first articulating element formed of a ceramic material into a mold;
introducing a polymer material into the mold;
compressing the first articulating element and the polymer material to mold the polymer to the first articulating element to create a first ceramic-polymer interface and to form the first articular component; and
manufacturing a second articular component by
placing a second articulating element formed of a ceramic material into a mold;
introducing a polymer material into the mold;
compressing the second articulating element and the polymer material to mold the polymer to the second articulating element to create a second ceramic-polymer interface and form the second articular component, wherein the first and second articulating elements are configured to cooperate to provide articulation to the prosthetic device.

33. The method of claim 32, including roughening at least a portion of the first ands second articulating elements.

34. The method of claim 32, including generating porous features on the polymer that promote bone ingrowth.

35. The method of claim 32, including applying a bone-growth substance to the molded polymer.

36. The method of claim 32, including polishing articular surfaces of the first and second articulating elements to allow reduced friction articulation.

37. The method of claim 32, including applying surface features to at least one of the first and second articular components, the surface features being configured to engage one of the vertebral bodies.
